(19) United States
(12) Patent Application Publication
(10) Pub. No.: US 2026/0248894 A1
(43) Pub. Date: Aug. 27, 2026

(54) COMBINATION THERAPY FOR PRETERM INFANTS

(71) Applicant: Oak Hill Bio Ltd, Altrincham (GB)

(72) Inventors: Nazila Salamat-Miller, Arlington, MA (US); Norman Barton, Phoenix, MD (US)

(21) Appl. No.: 18/862,222

(22) PCT Filed: May 3, 2023
(86) PCT No.: PCT/EP2023/061721

§ 371 Date: Nov. 1, 2024

Related U.S. Application Data
(60) Provisional application No. 63/338,221, filed on May 4, 2022.

(51) Int. Cl. A61K 38/30; A61K 31/137; A61K 31/4196; A61K 31/4468; A61K 31/485; A61K 31/522; A61K 31/5517; A61K 31/7036; A61K 38/14; A61K 38/28

(52) U.S. Cl. CPC A61K 38/30; A61K 31/137; A61K 31/4196; A61K 31/4468; A61K 31/485; A61K 31/522; A61K 31/5517; A61K 31/7036; A61K 38/14; A61K 38/28

(57) ABSTRACT

The present disclosure relates to a method of treatment or prophylaxis of a preterm infant by administering intravenously a therapeutic amount of a composition comprising IGF-1 and IBGBP-3 in combination with a therapy selected from the group comprising: caffeine, fentanyl, fluconazole, gentamicin, insulin, midazolam, morphine, low dose norepinephrine, vancomycin concentrations of 5 mg/ml or less, parenteral nutrition (or a composition for the same use) and combinations thereof.

The present disclosure relates to a combination therapy comprising IGF-1 and IBGBP-3, for example as a complex, with compatible drugs/medicines and/or excluding incompatible drugs/medicines.

### BACKGROUND

The preterm infants as a patient population are some of the most delicate, vulnerable and difficult to treat.

The rhIGF-1/rhIGFBP-3 drug product is the recombinant human (rh) version of the naturally occurring protein complex of insulin-like growth factor-1 (IGF-1) and its most abundant binding protein, insulin-like growth factor binding protein-3 (IGFBP-3). The product is currently under investigation for the prevention of complications of prematurity. It is administered parenterally, in particular by IV infusion. However, IV access can be a challenge in preterm infants in the neonatal intensive care unit (NICU), as they typically require multiple IV therapies, including different classes of medications and parenteral nutrition (PN).

Historically, there has been a lack of compatibility data available for drugs administered to preterm infants. A review of neonatal drug studies found that no documentation on compatibility was available for almost 60% of IV drug-drug infusions, and for 34% of IV drug-nutrition co-infusions administered in the neonatal intensive care unit.

As might be expected, there is a lack of comprehensive drug testing in neonates. One US study reported that only 35% of medications administered to neonates were approved by the US Food and Drug Administration for use in infants, and an Italian study found that 44% of medications were prescribed off-label in the preterm neonatal population. Such findings have potential implications for both treatment efficacy and safety in infants. The majority of compatibility studies are performed for small molecules co-administered with small molecules. In contrast literature was identified for compatibility testing for only two biologics, insulin and vasopressin, and standard biologic-specific testing methods have not been established.

rhIGF-1/rhIGFBP-3 is continuously infused and therefore compatibility with other medications is therefore of utmost importance.

The present inventors have established which commonly employed medicines can be co-administered with IGF-1 and IGF-BP-3 to preterm babies. This will likely improve safety and effectiveness of treatments, which is really important to the health and survival of these delicate patients.

### SUMMARY OF THE INVENTION

A summary of the invention is provided in the following paragraphs:
1. A method of treatment or prophylaxis of a preterm infant (also referred to a neonate herein) by administering intravenously a therapeutic amount of a composition comprising IGF-1 and IBGBP-3, for example as a complex, in combination with a therapy or therapies selected from the group comprising: caffeine (for example caffeine citrate), fentanyl, fluconazole, gentamicin, insulin, midazolam, morphine (for example sulfate), low dose norepinephrine, vancomycin at concentrations of 5 mg/ml or less, parenteral nutrition (for example intravenous fat emulsion, with or without electrolytes).
1A A composition of IGF-1 and IGFBP-3, for example as a complex, administered intravenously for the treatment or prophylaxis of a preterm infant in combination with a therapy or therapies selected from the group comprising: caffeine (for example caffeine citrate), fentanyl, fluconazole, gentamicin, insulin, midazolam, morphine (for example sulfate), low dose norepinephrine, vancomycin at concentrations of 5 mg/ml or less, parenteral nutrition (for example intravenous fat emulsion, with or without electrolytes).
1B A composition of IGF-1 and IGFBP-3, for example as a complex, for use in the manufacture of a medicament for the treatment or prophylaxis by intravenous administration of a preterm infant employed in combination with a therapy or therapies selected from the group comprising: caffeine (for example caffeine citrate), fentanyl, fluconazole, gentamicin, insulin, midazolam, morphine (for example sulfate), low dose norepinephrine, vancomycin at concentrations of 5 mg/ml or less, parenteral nutrition (for example intravenous fat emulsion, with or without electrolytes).
2. A method of treatment or prophylaxis of a preterm infant (also referred to a neonate herein) by administering intravenously a therapeutic amount of a composition comprising IGF-1 and IBGBP-3, for example as complex, in combination with one or more further therapies, [for example selected from the group comprising: caffeine (for example caffeine citrate), fentanyl, fluconazole, gentamicin, insulin, midazolam, morphine (for example sulfate), intravenous fat emulsion, parenteral nutrition (for example intravenous fat emulsion, with or without electrolytes)],
   wherein the further therapy is not co-administration of a therapy selected from amikacin sulfate, ampicillin sodium, dobutamine (for example hydrochloride), dopamine (hydrochloride), flurosemide, meropenem, high dose of norepinephrine, penicillin G, vancomycin at concentrations of over 5 mg/ml and combinations thereof.
2A A composition of IGF-1 and IGFBP-3, for example as a complex, for the treatment or prophylaxis by intravenous administration of a preterm infant employed in combination with one or more further therapies,
   [for example selected from the group comprising: caffeine (for example caffeine citrate), fentanyl, fluconazole, gentamicin, insulin, midazolam, morphine (for example sulfate), intravenous fat emulsion, parenteral nutrition (for example intravenous fat emulsion, with or without electrolytes)],
   wherein the further therapy is not co-administration of a therapy selected from amikacin (sulfate), ampicillin (sodium), dobutamine (for example hydrochloride), dopamine (hydrochloride), flurosemide, meropenem, high dose of norepinephrine, penicillin G, vancomycin (vancomycin (infused at concentration 50 mg/mL or less) thereof.
2B A composition of IGF-1 and IGFBP-3, for example as a complex, for use in the manufacture of a medicament for the treatment or prophylaxis by intravenous administration of a preterm infant employed in combination with one or more further therapies,
   [for example selected from the group comprising: caffeine (for example caffeine citrate), fentanyl, fluconazole, gentamicin, insulin, midazolam, morphine sulfate, intravenous fat emulsion, parenteral nutrition (for example intravenous fat emulsion, with or without electrolytes)],
   wherein the further therapy is not co-administration of a therapy selected from amikacin (sulfate), ampicillin (sodium), dobutamine (for example hydrochloride), dopamine (hydrochloride), flurosemide, meropenem, high dose of norepinephrine, penicillin G, vancomycin (infused at concentration 50 mg/mL or less).
3. A method or composition according to any preceding paragraph, wherein the combination therapy is co-administered (for example is delivered via the same IV line).
4. A method or composition according to any preceding paragraph, wherein the IGF-1 and IBGBP-3 is administered in combination with caffeine (for example caffeine citrate)
5. A method or composition according to any preceding paragraph, wherein the IGF-1 and IBGBP-3 is administered in combination with fentanyl.
6. A method or composition according to any preceding paragraph, wherein the IGF-1 and IBGBP-3 is administered in combination with fluconazole.
7. A method or composition according to any preceding paragraph, wherein the IGF-1 and IBGBP-3 is administered in combination with gentamicin.
8. A method or composition according to any preceding paragraph, wherein the IGF-1 and IBGBP-3 is administered in combination with insulin.
9 A method or composition according to any preceding paragraph, wherein the IGF-1 and IBGBP-3 is administered in combination with midazolam.
10. A method or composition according to any preceding paragraph, wherein the IGF-1 and IBGBP-3 is administered in combination with morphine (for example sulfate).
11. A method or composition according to any preceding paragraph, wherein the IGF-1 and IBGBP-3 is administered in combination with low dose of norepinephrine.
12. A method or composition according to any preceding paragraph, wherein the IGF-1 and IBGBP-3 is administered in combination with vancomycin wherein the latter is infused at concentrations of 5 mg/mL or less, such as less than 5 mg/mL.
13. A method or composition according to any preceding paragraph wherein, wherein the IGF-1 and IBGBP-3 is administered in combination with vancomycin and the latter is administered as an infusion and does not exceed 50 mg/Ml.
14. A method or composition according to any preceding paragraph, wherein the IGF-1 and IBGBP-3 is administered in combination with parenteral nutrition (for example intravenous fat emulsion, with or without electrolytes).
15. A method or composition according to any one of paragraph 2 to 14, wherein the further non-co-administered therapy is administered by a different IV line or by a different route of administration, such as intramuscular administration.
16. A method or composition according to any one of paragraph 2 to 14, wherein the further non-co-administered therapy is omitted completely.
17. A method or composition according to any preceding paragraph, wherein preterm infants are in the range 23 to 34 weeks post gestation, when treatment is initiated.
18. A method or composition according to any preceding paragraph, wherein the preterm infants are administered IGF-1 and IGFBP3 by infusion, for example continuous infusion, in particular for at least 1 week, for example 2 to 6 weeks, such as 2, 3, 4, 5 or 6 weeks.
19. A method or composition according to claim 18, wherein the infusion is initiated within 24 hours of birth, for example within 1, 2, 3, 4, 5, 6, 7, 8, 9, 10, 11, 12, 13, 14, 15 16, 17, 18, 19, 20, 21, 22, 23 and 24 hours of birth, in particular within 1 hour of birth.
20. A method or composition according to any one of paragraphs 1 to 19, where the composition comprises equimolar amounts of IGF-1 and IGFBP-3.
21. A method or composition according to any preceding paragraph, wherein 200 to 500 μg/Kg/24 hours of the IGF-1 and IGFBP-3 complex are administered.
22. A method or composition according to any preceding paragraph, wherein the 55 to 110 μg/Kg/24 hours of IGF-1 are administered.
23. A method or composition according to any preceding paragraph, wherein serum levels of IGF-1 are maintained with the range 28 to 109 ng/ml.
24. A method or composition according to any one of paragraph 1 to 23, wherein a low dose of norepinephrine is 0.05 μg/kg/min or less.
25. A method or composition according to any one of paragraphs 1 to 23, wherein the pH of the composition comprising IGF-1 and IGFBP3 is 5.5±~0.3, for example wherein the pH is within the range 5.2 to 5.8.

Non-combination-therapy are incompatible or unstable for formulation with or delivery in admixture with IGF-1/IGFBP-3.

“Not co-administered” as employed herein refers to: NOT administered by the same route or NOT administered (what-so-ever), in particular NOT administered.

IGF-1/rhIGFBP-3 therapy as employed herein is recombinant human (rh) insulin-like growth factor-1 (IGF-1) and, insulin-like growth factor binding protein-3 (IGFBP-3), generally as a complex, more specifically complexed in a range 0.75 to 1.25:1 or 1:0.75-1.25, for example as a 1:1 complex.

In one embodiment the IGF-1/IGFBP-3 therapy is continuously administrated.

In one embodiment the combination therapy is continuously administrated.

Therapy as employed herein includes parenteral feeding.

“In combination” (also referred to as combination therapy) as employed herein refers to co-formulation, admixed or co-administered, in particular co-administered by the same route.

“Generally, unless the context indicates otherwise, in combination refers to a compatible combination”.

“Compatible combination” as employed herein refers to a agents/ingredients/components that are suitable for delivery/administration in combination, for example the components are at least physically and chemically stable when admixed and/or co-formulated.

Co-administered as employed herein refers to delivery/administration at the same time, in particular by the same route, such as IV, especially via the same IV line. Thus the co-administered medicaments may be present contemporaneously, for example in an IV and/or IV bag.

In one embodiment co-administered medicaments are in separate formulations.

Co-formulated as employed herein refers to a single pharmaceutical formulation comprising two or more medicaments.

Admixture as employed herein refers to formulations/medicaments mixed at the time of administration or shortly before, i.e. mixed extemporaneously. The latter includes mixing in an IV line or bag, for example by adding a medicament to one already being dispensed.

In one embodiment combination therapy as employed herein refers to a therapy that is compatible for delivery with IGF-1/IGFBP-3, in particular stable when delivered in the same formulation, in admixture or via the same route (such as the same IV lines).

In one embodiment the combination therapies employed in the present disclosure are delivered in admixture, i.e. together, concomitantly. What is more the stability of the combination medicaments is adequate to allow this.

In one embodiment the compatible drug/medicament is administered over a short period, for example 1 to 30 mins.

In embodiment the compatible drug/medicament is administered over the same period of time as the IGF-1/IGFBP-3.

In one embodiment the stability is physical stability.

In one embodiment the stability is chemical stability.

In one embodiment the activity of the entity (such as IGF-1/IGFBP-3 and or other medicament(s)) is stable, in particular the activity is not diminished.

In one embodiment the medicaments are thermally stable, for example at storage temperatures such as 4° C. to room temperature.

In one embodiment the stability is concentration dependent.

Changes in physical stability include aggregation, flocculation, particulates crashing out of solution or the like.

Changes in chemical stability include degradation, changes in surface charge, changes in pH or similar.

The non-combination-therapies are generally not stable in admixture with IGF-1/IGFBP-3. In some embodiments, where it is available, then the further therapy can be administered separately, for example from a separate IV line or subcutaneously. Alternatively, administration of IGF-1/IGFBP-3 may be halted to allow administration of non-combination-therapy. In one embodiment administration of IGF-1/IGFBP-3 is resumed after administration of non-combination-therapy. If this is not available then it administration of the non-compatible therapy should be avoided.

In one aspect the present invention employs a formulation of IGF-1 and IGFBP-3 described in WO2022/086953, incorporated herein by reference. The parameters of the formulation disclosed may be employed as basis for amendments to the present claims.

In some embodiment the present disclosure employs a pharmaceutical composition comprising a protein complex comprising recombinant insulin-like growth factor 1 (rIGF-1), recombinant insulin-like growth factor binding protein 3 (rIGFBP-3), and a surfactant at a concentration of between about 0.001% to 2.4% v/v, (such as 0.0025% and 0.0075%, in particular about 0.005%) wherein the rIGF-1 and rIGFBP-3 are complexed in a range of 0.75 to 1.25:1 or 1:0.75-1.25, for example, in equimolar amounts.

In some embodiments the surfactant is polysorbate surfactant, for examples polysorbate 20 (also known as polyoxyethylene sorbitan monolaurate) or polysorbate 80 (also known as polyoxyethylene sorbitan monooleate).

In some embodiments, the polysorbate surfactant is polysorbate 20.

In some embodiments, the polysorbate surfactant is polysorbate 80.

In some embodiments, polysorbate surfactant is at a concentration of between about 0.001% to 2.4% v/v, for example at a concentration of between about 0.2% to 0.4%.

In some embodiments, polysorbate surfactant is at a concentration of about 0.001%, 0.002%, 0.003%, 0.004%, 0.005%, 0.01%, 0.02%, 0.03%, 0.05%, 0.10%, 0.15%, 0.2%, 0.5%, 0.7%, 1.0%, 1.2%, 1.5%, 1.8%, 2.0%, 2.2% or 2.4% v/v.

In some embodiments, the polysorbate surfactant is at a concentration of between about 0.0025% and 0.0075%, such as about 0.0025%, about 0.005%, or about 0.0075% v/v, in particular about 0.005% v/v.

In one aspect the present disclosure relates to a KIT comprising a combination therapy according to the present disclosure.

In one embodiment high dose norepinephrine is 2.0 μg/kg/min or more.

In one embodiment high dose norepinephrine is 1.0 μg/kg/min or more.

In one embodiment high dose norepinephrine is more than 0.05 μg/kg/min.

In one embodiment low dose norepinephrine is 1.0 μg/kg/min or less.

In one embodiment there medicament compatibility data is disclosed on the drug packaging of IGF-1/IGFBP-3, a leaflet associated therewith, a website with product information for the same or the like.

In one embodiment the IV administration according to the present disclosure is employed in combination with surfactant therapy. Surfactants, also known as rescue surfactant, include beractant.

“Is” as employed herein means comprising.

In the context of this specification “comprising” is to be interpreted as “including”.

Embodiments of the invention comprising certain features/elements are also intended to extend to alternative embodiments “consisting” or “consisting essentially” of the relevant elements/features.

Where technically appropriate, embodiments of the invention may be combined.

Technical references such as patents and applications are incorporated herein by reference.

Any embodiments specifically and explicitly recited herein may form the basis of a disclaimer either alone or in combination with one or more further embodiments.

The background section may be used as basis for an amendment.

The present application claims priority from U.S. 63/338,221 filed 4 May 2022, incorporated herein by reference especially the sequences, which are explicitly incorporated herein. The priority document may be employed to correct errors in the present specification.

The present invention is further described by way of illustration only in the following examples, which refer to the accompanying Figures.

### BRIEF DESCRIPTION OF THE FIGURES

FIG. 1 Risk assessment design. RP-HPLC reversed-phase high-performance liquid chromatography, SEC-HPLC size-exclusion high-performance liquid chromatography, USP United States Pharmacopeia

FIG. 2-7 Shows HPLC plots

### EXAMPLES

In this study the physical compatibility of the rhIGF-1/rhIGFBP-3 drug product when mixed with frequently administered medications, was investigated. To generate a complete picture of compatibility, chemical compatibility at the level of the small-molecule content was evaluated as part of a separate study on the protein content/chemical modification.

The work was conducted to systematically evaluate and build a comprehensive body of data on the compatibility of rhIGF-1/rhIGFBP-3 with commonly administered intravenous drugs to aid clinicians' decision-making regarding the co-infusion of rhIGF-1/rhIGFBP-3.

Drug compatibility testing studies should be conducted as early as feasible in the investigational phase of a neonatal drug to allow sufficient time for study findings to inform clinical trials as well as the eventual adoption of the drug in clinical practice.

### 1.0 TEST MEDICATIONS

Test medications were selected on the basis of clinical priority (i.e., medications frequently administered to neonates), as identified by clinical experts and from investigative sites for the phase 2 trial. The 19 medications included in this study were amikacin, ampicillin, caffeine citrate, dobutamine, dopamine, fentanyl citrate, fluconazole, furosemide, gentamicin, insulin, intravenous fat emulsion, meropenem, midazolam, morphine sulfate, norepinephrine bitartrate, penicillin G, custom-mixed PN solution (with and without electrolytes), PN solution+intravenous fat emulsion, and vancomycin (Table 1 below). Most of the test medications were small-molecule drugs, whereas rhIGF-1/rhIGFBP-3 (Takeda, Lexington, MA, USA) is a recombinant protein.

TABLE 1
Medications tested in the compatibility study

| Medication | Manufacturer |
|---|---|
| Amikacin sulfate | Teva Parenteral Medicines, Irvine, CA, USA |
| Ampicillin | Sandoz Inc., Princeton, NJ, USA |
| Caffeine citrate | Sagent Pharmaceuticals, Schaumburg, IL, USA |
| Dobutamine HCl | Baxter, Deerfield, IL, USA |
| Dopamine HCl | Hospira Inc., Lake Forest, IL, USA |
| Fentanyl citrate | Hospira Inc., Lake Forest, IL, USA |
| Fluconazole | Sagent Pharmaceuticals, Schaumburg, IL, USA |
| Furosemide | Hospira Inc., Lake Forest, IL, USA |
| Gentamicin | APP Pharmaceuticals, Los Angeles, CA, USA |
| Insulin (Novalin R) | Novo Nordisk, Bagsvrd, Denmark |
| Intravenous fat emulsion (Intralipid ® 20%) | Fresenius Kabi, Bad Homburg, Germany |
| Meropenem | Hospira Inc., Lake Forest, IL, USA |
| Midazolam HCl | Akorn, Lake Forest, IL, USA |
| Morphine sulfate | Hospira Inc., Lake Forest, IL, USA |
| Norepinephrine bitartrate (Levophed) | Hospira Inc., Lake Forest, IL, USA |
| Penicillin G potassium | Sandoz Inc., Princeton, NJ, USA |
| PN solution (with and without electrolytes)^{a} | Solutions were prepared by Tufts University, |
| | Medford, MA, USA |
| PN solution + Intravenous fat emulsion | Solutions were prepared by Tufts University and |
| (Intralipid 20%)^{a} | mixed in house |
| Vancomycin HCl | Hospira Inc.; Mylan, Canonsburg, PA, USA |

PN parenteral nutrition
^{a}Solutions were studied within 24 h of preparation.

### 2.0 RISK ASSESSMENT DESIGN AND OVERVIEW

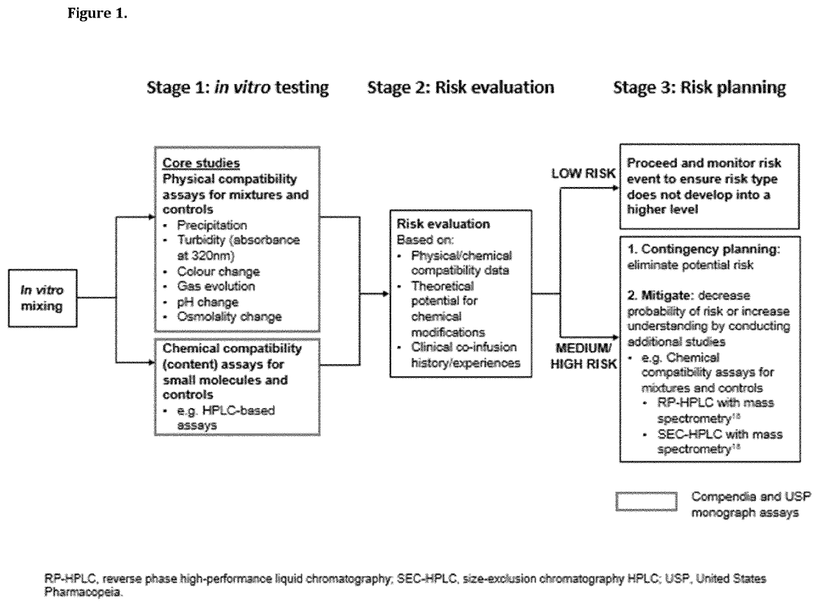

The risk assessment methodology was developed by a cross-functional team comprising clinicians, neonatal pharmacists, and representatives from the study sponsor's product development departments for small molecules, biologics, clinical, and clinical operations. The risk assessment comprised of three consecutive stages (FIG. 1): (1) in vitro testing to determine the physical and chemical compatibility of rhIGF-1/rhIGFBP-3 with other medications (small molecules only); (2) a risk evaluation for each of the test medications, taking into account the known theoretical potential for chemical modifications, proximity to the isoelectric point of the protein when not in the mixture (based on pH value and probability of chemical modification), and the clinical co-infusion history (including co-administration with insulin, which shares a large homology with rhIGF-1); and (3) risk planning, based on an assessment of low, medium, or high risk of incompatibility.

### 3.0 MIXING PROTOCOLS

A mixing model was developed whereby rhIGF-1/rhIGFBP-3 and the test medication were mixed at one or more representative clinical doses. For all studies, the mixing calculations were performed with appropriate normalizations (time and neonates' weight) to devise a volume-based scheme. For these calculations, a dose of 250 μg/kg/24 hours (the dose used in the phase 2 trial) and at least two bracketing doses of the small-molecule medication were used (Table 2). For each study, multiple controls were designed to represent post mixing concentrations and matrices of either rhIGF-1/rhIGFBP-3 or the test medication. The controls were created by diluting the rhIGF-1/rhIGFBP-3 drug product either with its own formulation buffer (to create a concentration control) or with the matrix of the small molecule to study the effect of change in the absence of any small molecule.

Where applicable, the mixing duration was based on the calculated average infusion rate of rhIGF-1/rhIGFBP-3 (e.g., 250 μg/kg/24 hours dose normalized for a 0.5 kg neonate and the target rhIGF-1/rhIGFBP-3 protein concentration of 50 μg/mL) with each test medication at the highest dose (normalized for the same weight) and an estimated volume for an umbilical catheter (Table 2). In all studies, periodic sampling of the mixture and control solutions was performed. Additionally, longer mixing durations were considered as worst-case scenarios (e.g., interruptions could occur in clinical practice, extending the duration of the infusion) and to observe the continuation of any observed phenomena that occurred at the onset of mixing.

Two administration scenarios were assumed for the mixing duration calculations, where applicable. The first scenario was when each of the two medications were administrated via a pump, where the average of the two flow rates at the highest small-molecule dose was selected. In the second scenario, only rhIGF-1/rhIGFBP-3 was pumped, while the test medication was infused over a relatively short time (~15-30 minutes). For these scenarios and any other medications that were not pumped or infused, periodic observations and sampling occurred at selected time points (Table 2).

TABLE 2
rhIGF-1/rhIGFBP-3 drug product and small-molecule compatibility studies

| | | Preparation | | |
|---|---|---|---|---|
| | Mixing dose | matrix per | Time point for | |
| | for test | package | analysis (visual, | |
| Test medication^{a} | medication | insert | pH, OD320)^{b} | Notes |
| Amikacin sulfate | 15 and 18 | D5W | 0, 60, 120 min; | Osmolality testing at 0, |
| | mg/kg/30 min | | 24 h | 60, 120 min, and 24 h |
| Ampicillin sodium | 25 and 100 | WFI followed | 0, 30, 90 min; 3, 5, | |
| | mg/kg/5 min | by D5W | 8 h | |
| Caffeine citrate | 20 mg/kg/20 | N/A | 0, 60, 120 min; | Osmolality testing at 0, |
| | min | | 24 h | 120 min, and 24 h |
| Dobutamine | 2, 10, and 25 | N/A | 0, 60, 90 min | |
| hydrochloride in | μg/kg/min | | | |
| dextrose | | | | |
| Dopamine | 2, 5, 10, and 20 | N/A | 60 min | Osmolality testing was |
| hydrochloride | μg/kg/min | | | not part of the testing |
| | | | | panel |
| Fentanyl citrate | 0.5, 2, and 5 | D5W | 0, 60, 90 min | |
| | μg/kg/h | | | |
| Fluconazole | 6 and 12 | N/A | 0, 60, 120 min; | Osmolality testing at 0, |
| | mg/kg/30 min | | 24 h | 60, 120 min, and 24 h |
| Furosemide | 0.5, 1.0, and 2.0 | N/A | 0, 30, 60 min; 24 h | The observed turbidity |
| | mg/kg/5 min | | | was not associated with |
| | | | | any furosemide content |
| | | | | for up to 24 h, when |
| | | | | assessed by RP-HPLC |
| | | | | assay |
| Gentamicin | 1, 3, and 5 | N/A | 0, 60, 120 min; | |
| | mg/kg/30 min | | 24 h | |
| Insulin | 0.01, 0.05, and | NS | 0, 60, 90 min | |
| | 0.1 units/kg/h | | | |
| Intravenous fat | 0.5 and 3 | N/A | 0, 30, 60, 90 min | |
| emulsion | g/kg/day | | | |
| Meropenem | 10, 20, and 30 | D5W | 0, 15, 30, 60 min | |
| | mg/kg/30 min | | | |
| Midazolam | 20 and 60 | D5W | 0, 60, 90 min | |
| hydrochloride | μg/kg/h | | | |
| Morphine sulfate | 5, 10, and 50 | D5W | 0, 60, 90 min | |
| | μg/kg/h | | | |
| Norepinephrine | 0.05, 1.0, and | D5W | 0, 60, 120 min; | |
| bitartrate | 2.0 μg/kg/min | | 24 h | |
| Penicillin G | 25,000, 50,000, | | | |
| | and 125,000 | | | |
| | units/kg/30 | | | |
| | min | | | |
| PN solution + | 0.5 and 3 | N/A | 0, 30, 60, 90 min | |
| intravenous fat | g/kg/day with | | | |
| emulsion | 10 mL/h PN | | | |
| PN solution | 1, 4, and 10 | N/A | 0, 30, 60 min | Osmolality testing was |
| (with & without | mL/h | | | not part of the testing |
| electrolytes) | | | | panel |
| dextrose, 100 g/L | | | | |
| Vancomycin | 15 and 25 | WFI | 0, 60, 120 min; | Conducted as a worst- |
| hydrochloride | mg/kg/60 min | | 24 h | case study where a |
| | | | | stock concentration of |
| | | | | 50 mg/mL vancomycin |
| | | | | was used for mixing |
| | | | | studies |
| | 15 and 25 | WFI followed | 0, 60, 90 min | A stock concentration |
| | mg/kg/24 h | by D5W | | of 5 mg/mL |
| | | | | vancomycin was used |
| | | | | for mixing studies |

D5W 5% dextrose in water, IGF-1 insulin-like growth factor-1, IGFBP-3 insulin-like growth factor binding protein-3, N/A not applicable (no preparation/reconstitution/dilution was needed), NS normal saline, OD320 optical density at 320 nm, PN parenteral nutrition, rh recombinant human, RP-HPLC reversed-phase high-performance liquid chromatography, WFI, water for injection
^{a}The medications were prepared per each package insert; when applicable, each medication was diluted with the recommended diluents (0.9% normal saline, 5% dextrose and sterile water for injection).
^{b}Osmolality was tested at 60, 90, and 120 minutes and at 24 hours except where otherwise specified.

### 4.0 PHYSICAL COMPATIBILITY

Physical compatibility assays were compared for test samples and corresponding control solutions. In line with the existing literature, we used the following methods, or modified versions thereof, to assess the physical compatibility of rhIGF-1/rhIGFBP-3 with the co-infused drugs: visual observation (United States Pharmacopeia [USP]<790>), optical density at 320 nm (USP <851> and <857>), pH measurements (USP <791>), and osmolality (USP <785>) at room temperature.

The aim of visual observation was to determine the presence of any precipitation, visible particulates, and flocculent matter, as well as any color change (compared with water) and/or gas formation, which are potential indicators of chemical modification(s).

All vials for physical compatibility testing were examined under the same lighting conditions: against a white and black background using both fluorescent light and Tyndall light (Spectralight III, Macbeth/X-Rite, Grand Rapids, MI, USA or MIH-DX, Bosch/Eisai Machinery, Waiblingen, Germany). Mixture and control samples were analyzed for appearance post mixing at specified time points (Table 1). Optical density measurements at 320 nm were carried out using an ultraviolet-visible spectrophotometer (SpectraMax M5, Molecular Devices, San Jose, CA, USA) for the detection of turbidity, an indicator of submicroscopic protein aggregation. Measurements were performed in triplicate using a 1-cm path-length quartz cuvette for each sample at each time point, and the average of these was recorded. pH values were recorded for each solution in triplicate using a calibrated pH meter (Model 215, Denver Instrument, Bohemia, NY, USA; Fischer Scientific Accumet XL150, Pittsburgh, PA, USA), and the average was reported. Osmolality changes post mixing at room temperature were recorded using a calibrated osmometer (Model 3250, Advanced Instruments, Norwood, MA, USA). Triplicate readings were ascertained, and the average was recorded. Test medications were considered physically compatible with rhIGF-1/rhIGFBP-3 if there was no observed change in color, precipitation, turbidity, or gas evolution, or if there was no clinically relevant change in osmolality or pH.

Any change ±~0.3 pH of the mixtures from that of the rhIGF-1/rhIGFBP-3 drug product control (pH 5.5) was considered a change that could impact rhIGF-1/rhIGFBP-3 quality, in which case the small-molecule medication would be considered not compatible and would require further protein-specific data for evaluation. The considered range was based on the control and release specification of rhIGF-1/rhIGFBP-3 (5.5±0.3) and a priori knowledge of the potential degradation and known stability of the rhIGF-1/rhIGFBP-3 drug product. The changes in osmolality values were considered using a less stringent criterion, owing to existing clinical practices and in consideration of release specifications of rhIGF-1/rhIGFBP-3 (300±30 mOsmol/kg).

### 5.0 SMALL-MOLECULE CHEMICAL COMPATIBILITY

The concentration of small-molecule test medications post mixing was assessed using either reversed-phase high-performance liquid chromatography (RP-HPLC), with ultraviolet (UV) detection, or ion chromatography with electrochemical detection at the last specified time point(s) (Table 2) (USP monographs or modified versions); for example while a RP-HPLC-UV method was used for the detection of majority of molecules, for some, such as Amikacin and Gentamicin, a modified version of the USP ion chromatography assay with an electrochemical detection was used. For each medication, a qualification of the USP methods was conducted to ensure specificity, linearity, repeatability, and accuracy of the method. Example chromatograms for one small molecule (Gentamicin) are presented in the Supplementary information. Chemical incompatibility was considered to be a loss of the small-molecule content of ~10% or more over the defined testing period. Small-molecule analysis was not possible for PN or lipids owing to the complex nature of such mixtures.

### 6.0 RHIGF-1/RHIGFBP-3 CHEMICAL COMPATIBILITY

Sensitive mass spectrometry-based protein-specific methodologies have been developed by Takeda to assess the chemical compatibility of the rhIGF-1/rhIGFBP-3 drug product. The development of protein-specific methodologies is reported separately.

### 7.0 RISK EVALUATION AND RISK PLANNING

A comprehensive risk evaluation was completed for medications where in vitro (non) compatibility was indicated. (See Risk assessment design and overview in Methods for a description of the risk evaluation). A risk event was defined as “rhIGF-1/rhIGFBP-3 is not compatible with the co-infused drug over the duration and condition of the simulated mixing studies”.

The risk evaluation was performed for each co-infused test medication to determine the probability and severity of a risk occurrence. Probability was defined as the likelihood of an effect on safety, efficacy, or quality; severity was defined as the severity of the impact should the risk event occur (Table 3). On the basis of the level of probability and severity (low, medium, or high), a risk planning strategy was developed for each medication (Table 4). The cross-functional team of subject matter experts performed the final assessments and endorsed the clinical recommendations.

TABLE 3
Risk assessment definition

| Description of risk | Description of risk event |
|---|---|
| Probability | What is the probability of the risk event occurring? Likelihood of an |
| | effect on safety, efficacy, or quality. |
| | High: High risk based on scientific rationale (e.g., pH changes over |
| | time for the mixing duration and conditions) |
| | Medium: Moderate probability of chemical modification (e.g., pH |
| | post mixing is outside of the demonstrated long-term pH range for |
| | stability, but all other observations are consistent with control |
| | samples) |
| | Low: Impact is not expected based on scientific rationale (e.g., |
| | mixture pH is within pH range acceptable for both rhIGF- |
| | 1/rhIGFBP-3 and the co-infused drug) |
| Severity | What will be the severity of the impact should the risk event occur? |
| | High: Major effect on patient safety and therapeutic or |
| | biotherapeutic efficacy and quality, and effects on the co- |
| | administrated drug efficacy and quality as demonstrated by |
| | physical incompatibility (e.g., precipitation) |
| | Medium: Moderate effect of chemical compatibility; loss of |
| | potency (content for small-molecule drugs) observed for the in- |
| | use duration and condition |
| | Low: No effect on the core testing based on biotherapeutic release |
| | specification (e.g., the drugs are physically compatible, with no |
| | observable loss of content) |

IGF-1 insulin-like growth factor-1, IGFBP-3 insulin-like growth factor binding protein-3, rh recombinant human.

TABLE 4
Risk prioritization grid with associated risk planning
Risk prioritization grid

| | | | | |
|---|---|---|---|---|
| SEVERITY | High | High severity | High severity | High severity |
| | | Low probability | Medium | High probability |
| | | | probability | |
| | Medium | Medium severity | Medium severity | Medium severity |
| | | Low probability | Medium | High probability |
| | | | probability | |
| | Low | Low severity | Low severity | Low severity |
| | | Low probability | Medium | High probability |
| | | | probability | |
| | | Low | Medium | High |

PROBABILITY

Risk planning

| | |
|---|---|
| Avoid/contingency | Change plan in order to eliminate potential risk |
| (H/H, H/M, M/H) | Contingency plan must be developed and in place |
| | To be implemented when event occurs or at trigger point |
| | Define trigger points for evaluation and ensure appropriate action |
| | is taken (e.g., activate contingency plan) |
| | Example: Avoid administrating the incompatible product. |
| | Communicate with clinical site on the risk of co-infusion. Switch to |
| | a compatible product |
| Avoid/mitigate | Change plan in order to eliminate potential risk or plan how to |
| (M/M, L/H, H/L) | minimize the impact (e.g., separate line for infusion and avoid co- |
| | infusion) |
| | Define/set trigger points and take appropriate action where |
| | necessary |
| | Examine each identified risk area |
| | Isolate the cause |
| | Develop response |
| | Example: Avoid administrating the incompatible product. Use a |
| | separate line for infusion |
| Passive acceptance | Accept and monitor risk to ensure risk type does not develop into a |
| (M/L, L/M, L/L) | higher level |
| | Example: Proceed with administration after collecting the initial |
| | physical data, augmented by a cross-functional risk-based |
| | assessment to support the co-administration |

H high, L low, M medium

### 8.0 RESULTS

### 8.1 In Vitro Physical Compatibility

Of the 19 medications tested, physical compatibility was established for rhIGF-1/rhIGFBP-3 with caffeine citrate, fentanyl, fluconazole, gentamicin, insulin, intravenous fat emulsion, midazolam, morphine sulfate, PN solution+intravenous fat emulsion, PN solution (with and without electrolytes), and vancomycin (when dosed from a 5 mg/mL solution) (Table 5). The following medications were considered incompatible with rhIGF-1: amikacin, ampicillin, dobutamine, dopamine, furosemide, meropenem, norepinephrine, penicillin G, and vancomycin (when dosed from a 50 mg/mL solution).

TABLE 5
Physical and small-molecule compatibility of the rhIGF-1/rhIGFBP-3
drug product and small-molecule test medications

| | | Chemical | |
|---|---|---|---|
| | | compatibility | |
| | | for | |
| | | small-molecule | |
| Test | Physical | content | |
| medication | compatibility | (RP-HPLC) | Notes |
| Amikacin | No | Yes | Mixtures demonstrated a decrease in pH of ~0.6 |
| sulfate | | | units compared with controls; difference |
| | | | of ~12% in the osmolality of mixture samples |
| | | | compared with controls |
| Ampicillin | No | Yes | Observed change in pH of rhIGF-1/rhIGFBP-3 |
| sodium | | | post mixing beyond the pH range that |
| | | | maintains stability; mixture pH was too close |
| | | | to the isoelectric point of rhIGF-1/rhIGFBP-3 |
| Caffeine citrate | Yes^{a} | Yes | pH of the mixed rhIGF-1/rhIGFBP-3 and |
| | | | caffeine samples at two different doses |
| | | | demonstrated ~1.0 unit difference compared |
| | | | with the control; osmolality of mixture |
| | | | samples was ~40% lower compared with the |
| | | | rhIGF-1/rhIGFBP-3 sample (attributable to |
| | | | differences in formulation matrices between |
| | | | rhIGF-1/rhIGFBP-3 and caffeine citrate); |
| | | | however, significant volume of protein-specific |
| | | | studies confirmed the compatibility (see |
| | | | footnote) |
| Dobutamine | No | Yes | Although the medication demonstrated |
| hydrochloride | | | physical compatibility (no precipitation, no |
| | | | visible particles, no turbidity, no color change, |
| | | | and no gas evolution), on the basis of gained |
| | | | knowledge using protein-specific assays in the |
| | | | presence of sodium (meta)bisulfiteb the |
| | | | medication is considered incompatible |
| Dopamine | No | Yes | A dopamine dose-dependent physical change |
| hydrochloride | | | (pH change) was observed, not concurrently |
| | | | with any other changes (no visible particles, no |
| | | | turbidity, no color change, and no gas |
| | | | evolution). Additionally, the protein-specific |
| | | | data demonstrated an impact on the rhIGF- |
| | | | 1/rhIGFBP-3 drug product in the presence of |
| | | | sodium (meta)bisulfite^{b} |
| Fentanyl | Yes | Yes | An osmolality decrease of ~19% was |
| | | | observed for fentanyl at the highest dose of 5 |
| | | | μg/kg/h, as compared with the rhIGF- |
| | | | 1/rhIGFBP-3 drug product (attributed to |
| | | | dilution of rhIGF-1/rhIGFBP-3 formulation |
| | | | buffer), but the decrease was not considered |
| | | | clinically significant |
| Fluconazole | Yes | Yes | |
| Furosemide | No | Yes | Cloudiness observed within ~30 min of |
| | | | mixing |
| Gentamicin | Yes | Yes | Osmolality decrease of ~74% was observed |
| | | | with the highest dose of 5 mg/kg/30 min but |
| | | | was not considered clinically significant |
| | | | because the low osmolality was observed for |
| | | | the gentamicin medication itself used at this |
| | | | clinically relevant dose |
| Insulin | Yes | Yes | Insulin monograph assay faced low recovery |
| | | | at low insulin doses; therefore, higher insulin |
| | | | concentrations were tested to obtain recovery |
| | | | and observe any incompatibility trends |
| Intravenous fat | Yes | N/A | No disruption of the emulsion, phase |
| emulsion | | | separation, color change, or lipid flocculation |
| | | | was observed upon mixing of the drug |
| | | | product and the fat emulsion (Intralipid ® |
| | | | 20%) |
| | | | pH increase of 0.2 observed after mixing |
| | | | rhIGF-1/rhIGFBP-3 drug product with |
| | | | intravenous fat emulsion (up to 90 min), due |
| | | | to the approximate pH value of 8.0 for the |
| | | | intravenous fat emulsion; the increase in pH |
| | | | of the mixture is within the drug stability pH |
| | | | specification so was not considered clinically |
| | | | significant |
| Meropenem | No | Yes | Observed pH change of ~2.4 post mixing, |
| | | | beyond the pH range that maintains the |
| | | | stability of rhIGF-1/rhIGFBP-3 |
| Midazolam | Yes | Yes | |
| Morphine | Yes | Yes | |
| sulfate | | | |
| Norepinephrine | Yes (only the | Yes | Only the lowest dose was physically |
| bitartrate | lowest dose)c | | compatible with respect to the pH change; |
| | | | however, the small-molecule medication is |
| | | | formulated with sodium (meta)bisulfite; |
| | | | therefore, we do not consider this drug to be |
| | | | compatible when formulated as such |
| Penicillin G | No | Yes | Further protein-specific investigations are |
| | | | ongoing to clarify the impact of the observed |
| | | | pH shift (up to 1.3 pH units for the highest |
| | | | penicillin G dose of 125,000 Units/kg/30 min) |
| | | | on rhIGF-1/rhIGFBP-3 |
| PN solution + | Yes | N/A | No pH change, disruption of the emulsion, |
| intravenous fat | | | phase separation, color change, or lipid |
| emulsion | | | flocculation was observed upon mixing of the |
| | | | drug product, PN (a high dose of 10 mL/h), |
| | | | and two doses of the fat emulsion |
| | | | (Intralipid ® 20%) |
| PN solution | Yes | N/A | No precipitation, particulate formation, or |
| (with and | | | mixture turbidity (as assessed visually and by |
| without | | | optical density at 320 nm) was observed |
| electrolytes) | | | No change in the pH of the final mixture of PN |
| | | | and drug product was observed |
| Vancomycin | Yes | Yes | Compatible only when dosed from a stock |
| hydrochloride | | | vancomycin concentration of 5 mg/mL; if |
| | | | infused at higher concentration (50 mg/mL), |
| | | | the change in pH is significant owing to the |
| | | | presence of hydrochloric acid in vancomycin's |
| | | | formulation |

IGF-1 insulin-like growth factor-1, IGFBP-3 insulin-like growth factor binding protein-3, N/A not applicable, PN parenteral nutrition, rh recombinant human, RP-HPLC reversed-phase high-performance liquid chromatography
^{a}To date, on the basis of these studies and protein-specific analyses, this medication has been removed from the incompatible list of medications. The protein-specific analyses demonstrated that the observed pH and osmolarity changes did not cause fragmentation, oxidation, aggregation, adduct formation, and generation of any free rhIGF-1 or rhlGFBP-3 submolecular units.
^{b}“Development of Protein-Specific Analytical Methodologies to Evaluate Compatibility of Recombinant Human (rh)IGF-1/rhIGFBP-3 with Intravenous Medications Co-Administered to Neonates” submitted for publication.
^{c}Compatible based on the physical data; however, the final compatibility decision is informed by other studies

8.2 Small-molecule chemical compatibility

Small-molecule compatibility was not affected post mixing for the medications tested. No loss of small-molecule content was observed for any of the medications tested in the mixture and corresponding controls (Table 1 above).

### 8.3 Risk Evaluation and Risk Planning

Risk evaluations were completed for all small-molecule test medications (except furosemide, where the studied mixture became turbid within ~30 minutes and clearly indicated incompatibility with the rhIGF-1/rhIGFBP-3 drug product). Where in vitro physical compatibility was confirmed, the subsequent risk evaluations confirmed a low probability and severity of an event within the context of in-use conditions. The risk of interaction or chemical modification, based on pH values, was also considered to be low for those medications showing in vitro compatibility.

Given the structural similarity between insulin and IGF-1, medication compatibility with insulin was given significant consideration when evaluating rhIGF-1/rhIGFBP-3 compatibility with tested medications. For example, compatibility with insulin has been established for midazolam and vancomycin, suggesting a low risk of incompatibility with rhIGF-1/rhIGFBP-3; however, particular attention was given to the small-molecule concentrations that were assessed in regard to compatibility with insulin. No compatibility data are available to date for insulin with fentanyl or fluconazole; however, on the basis of theoretical evaluation and the solution pH, a reaction is not expected.

Among the drugs that were observed to be incompatible with rhIGF-1/rhIGFBP-3 in the in vitro testing studies, the risk was classified as medium/high and appropriate actions were recommended.

TABLE 6
Methods: Chromatographic Conditions

| HPLC/Ion Chromatography | Dionex ICS-3000 | | |
|---|---|---|---|
| System: | | | |
| Detection Mode: | Integrated Amperometry | | |
| Mobile Phase: | Trifluoroacetic acid with pentafluoropropanoic acid, 50% | | |
| | Sodium Hydroxide (carbonate free), & acetonitrile, pH 2.6 | | |
| Post-column Reagent: | 20 g/L Sodium Hydroxide (Carbonate-free) | | |
| Flow Rate: | 1.0 mL/minute Mobile Phase (Pump 1) | | |
| | 0.3 mL/minute Post-column Reagent (Pump 2) | | |
| Column: | YMC Pack Pro C18 4.6 × 250 mm, 5 uM | | |
| Column Temperature: | 35° C. | | |
| Autosampler Temperature: | Ambient (not controlled) | | |
| Injector Volume: | 20 μL | | |
| Offset Level: | 10% | | |
| Range: | 300 uC | | |
| Run Time: | 50 minutes | | |
| | Time (seconds) | Potential (V) | Integration |
| Waveform: | 0.00 | 0.05 | |
| | 0.10 | 0.05 | On |
| | 0.40 | 0.05 | Off |
| | 0.41 | 0.75 | |
| | 0.55 | 0.75 | |
| | 0.56 | −0.15 | |
| | 1.00 | −0.15 | |

### 8.4 Small Molecule Chemical Compatibility

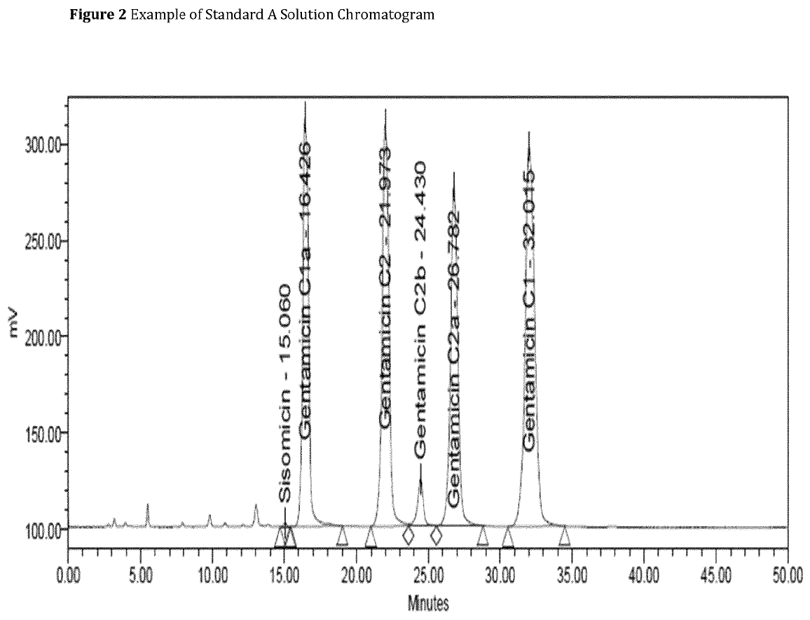

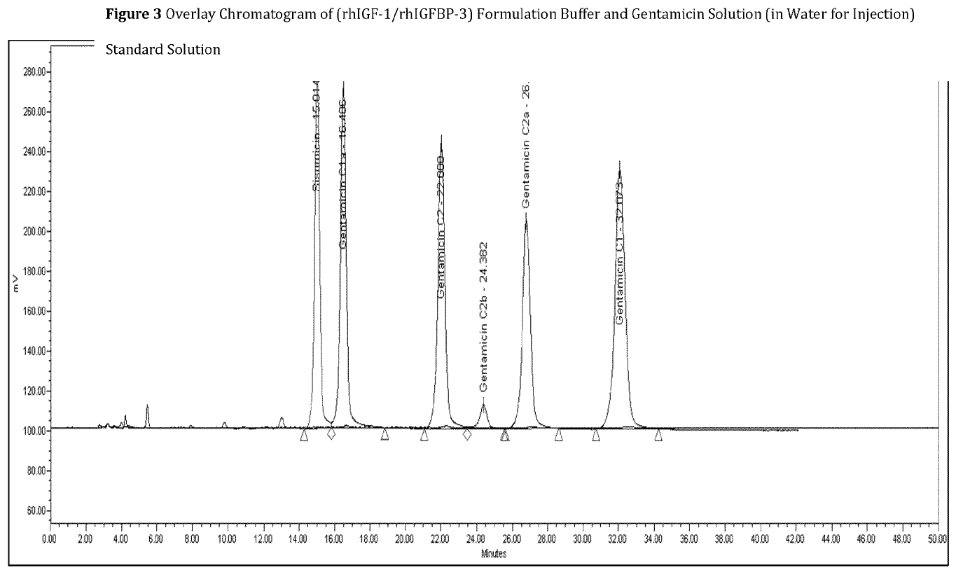

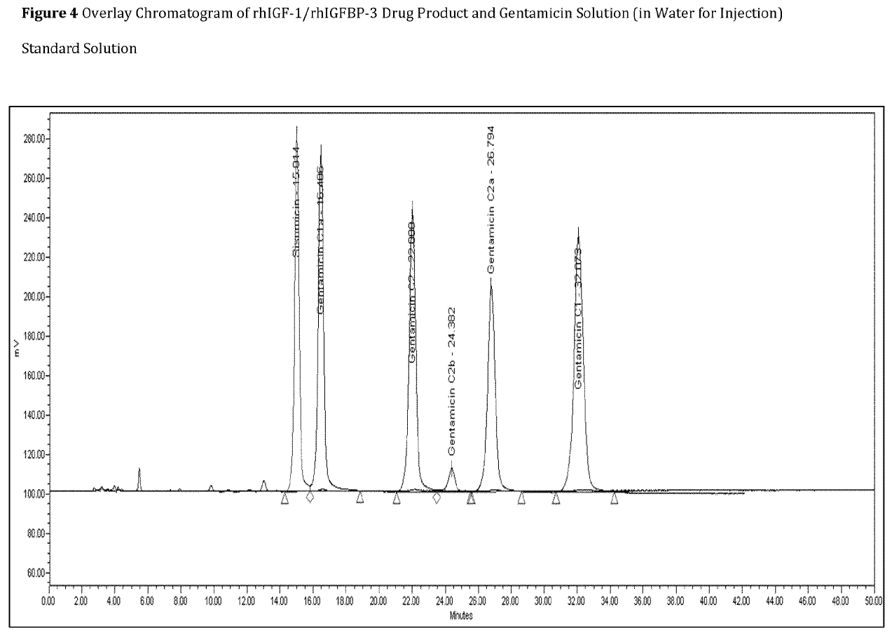

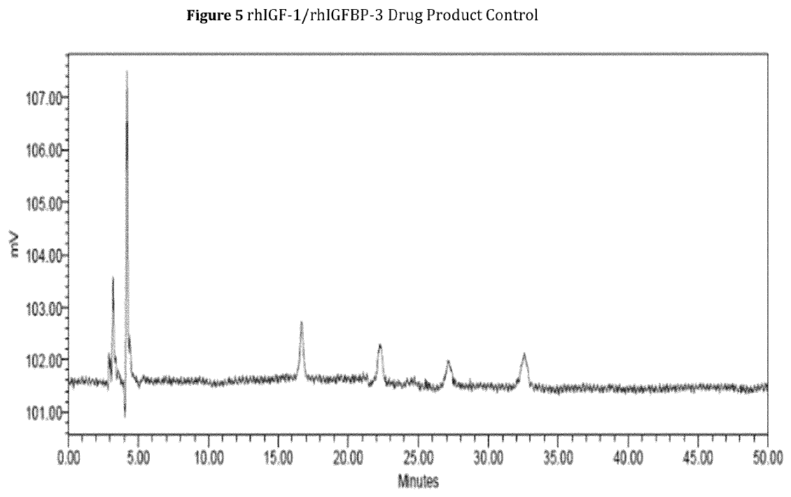

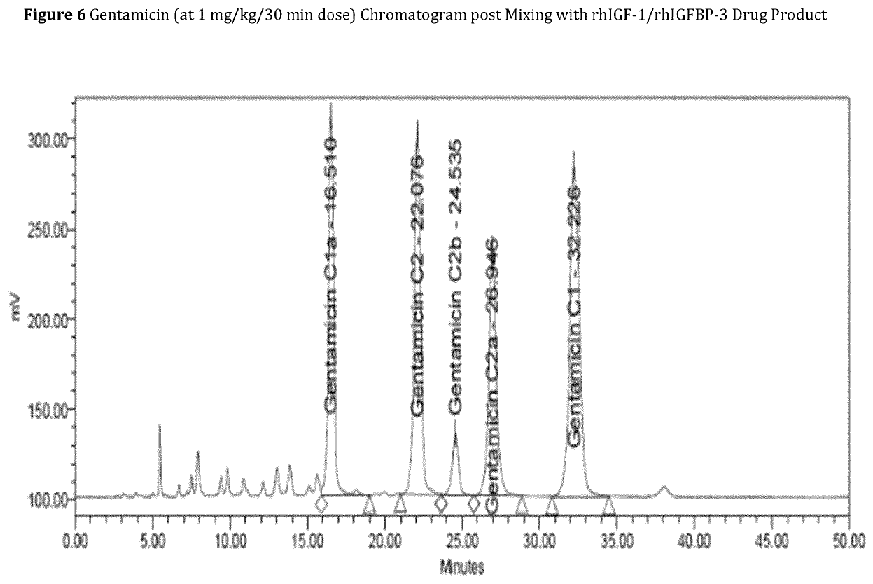

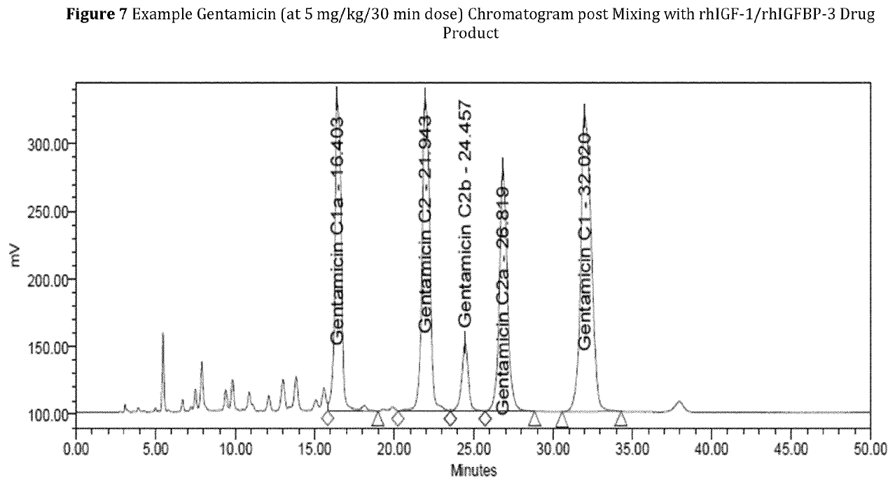

For each medication, a qualification of the USP methods (or its modified version) was conducted to ensure specificity, linearity, repeatability, and accuracy of the method. Gentamicin is presented in this section as an example. Amino sugar-driven antibiotics such as Gentamicin and its related substances were analyzed by ion chromatography with electro chemical detection. The USP monograph for the Content of Gentamicin in Gentamicin Sulfate was modified to quantitate the assay (concentration) of Gentamicin Sulfate. Several modifications were made to the USP monograph to obtain these results. Samples were quantitated using a n=5 standard calibration with a nominal standard concentration of 0.2 mg/mL Gentamicin Sulfate. The combined area (group) of all Gentamicin peaks was used to quantitate the amount of Gentamicin Sulfate (mg/mL) in samples. Mixture and control samples were then analyzed per the modified method to support the study. Mixture samples were diluted in mobile phase to obtain a suitable working concentration for all samples. Table 6 summarizes the analysis parameter for the presented example. FIGS. 2-7 show the representative chromatograms for Gentamicin.

### 9.0 CONCLUSIONS

In this study, in vitro testing indicated the physical compatibility of the rhIGF-1/rhIGFBP-3 drug product with 11/19 medications and nutritional therapies under the conditions and doses tested. For medications showing in vitro compatibility, the risk evaluation confirmed a low probability and severity of risk for incompatibility. Physical compatibility was not established with 8/19 medications. For drugs identified as incompatible, infusions would need to be redistributed to optimize available IV lines.

1. A method of treatment or prophylaxis of a preterm infant by administering intravenously a therapeutic amount of a:
composition comprising IGF-1 and IBGBP-3 complex by continuous infusion at a dose 200 to 500 μg/Kg/24 hours of complex initiated within 24 hours of birth;
in combination with a therapy selected from:
the group comprising: caffeine, fentanyl, fluconazole, gentamicin, insulin, midazolam, morphine, low dose norepinephrine, vancomycin concentrations of 5 mg/ml or less, parenteral nutrition and combinations thereof,
wherein the combination is co-administered and the components are at least physically and chemically stable when in admixture, such that the activity of the complex is not diminished, and the preterm infants are 23 to 34 weeks post gestation when treatment is initiated.

2-3. (canceled)

4. A method according to claim 1, wherein the IGF-1 and IBGBP-3 is administered in combination with caffeine (for example caffeine citrate).

5. A method according to claim 1, wherein the IGF-1 and IBGBP-3 is administered in combination with fentanyl.

6. A method according to claim 1, wherein the IGF-1 and IBGBP-3 is administered in combination with fluconazole.

7. A method according to claim 1, wherein the IGF-1 and IBGBP-3 is administered in combination with gentamicin.

8. A method er composition-according to claim 1, wherein the IGF-1 and IBGBP-3 is administered in combination with insulin.

9. A method according to claim 1, wherein the IGF-1 and IBGBP-3 is administered in combination with midazolam.

10. A method according to claim 1, wherein the IGF-1 and IBGBP-3 is administered in combination with morphine (for example sulfate).

11. A method to claim 1, wherein the IGF-1 and IBGBP-3 is administered in combination with low dose of norepinephrine.

12. A method according to claim 1, wherein the IGF-1 and IBGBP-3 is administered in combination with vancomycin wherein the latter is infused at concentrations less than 50 mg/mL.

13. A method according to claim 1, wherein the IGF-1 and IBGBP-3 is administered in combination with parenteral nutrition (for example intravenous fat emulsion, with or without electrolytes).

14-20. (canceled)

21. A method according to claim 1, wherein the preterm infants are administered IGF-1 and IGFBP3 for at least 1 week.

22. A method according to claim 21, wherein the IGF-1 and IGFBP3 is administered 2 to 6 weeks.

23. A method according to any claim 1, wherein the composition comprises equimolar amounts of IGF-1 and IGFBP-3.

24. A method according to claim 1, wherein the pH of the composition comprising IGF-1 and IGFBP-3 is in the range 5.2 to 5.8.

25. A method according to claim 1, wherein high dose norepinephrine is 2.0 μg/Kg/min or more.
